(19) United States
(12) Patent Application Publication
(10) Pub. No.: US 2014/0076311 A1
(43) Pub. Date: Mar. 20, 2014

(54) Face Mask for Administration of Gaseous Anesthesia

(71) Applicant: David J. Darab, Hickory, NC (US)

(72) Inventor: David J. Darab, Hickory, NC (US)

(21) Appl. No.: 13/838,966

(22) Filed: Mar. 15, 2013

Related U.S. Application Data
(60) Provisional application No. 61/702,047, filed on Sep. 17, 2012.

(51) Int. Cl. A61M 16/06; A61M 16/12; A61M 16/01

(52) U.S. Cl. CPC A61M 16/06; A61M 16/01; A61M 16/12
USPC 128/203.12

(57) ABSTRACT

An anesthesia administration system is provided that provides gaseous anesthesia to a patient's nose while providing access to a patient's mouth, the system comprising a face mask having an integrally formed gas inlet means, an integrally formed gas outlet means, and an integrally formed gas sampling means.

This application claims the benefit of Provisional Application No. 61/702,047, filed Mar. 17, 2012, which is incorporated herein by reference in its entirety.

### TECHNICAL FIELD OF THE INVENTION

The present invention relates to a face mask for use in providing gaseous anesthesia to a person in need thereof. More particularly the present invention relates to a face mask for use in providing gaseous anesthesia to a person need thereof while such person is undergoing a dental or medical procedure requiring access to the person's mouth.

### BACKGROUND OF THE INVENTION

The use of gaseous anesthesia in the medical and dental arts is well known. Such anesthesia is used to provide sedation of a patient during a medical, podiatric, or dental procedure that may be uncomfortable for a patient in full wakefulness. For example, nitrous oxide is a common gaseous anesthetic for use in certain dental procedures. The capability exists for trained medical professionals to provide such anesthesia in professional offices outside a hospital environment.

Thus most dental offices include an anesthesia administration system for administering gaseous anesthesia to a patient in need thereof. Such a system typically comprises a source of gaseous anesthesia, a face mask to be fitted over the nose of the patient, and means for conveying the gaseous anesthetic from the source to the face mask, where it will be inhaled by the patient.

Such gaseous anesthesia administration systems may present several issues for a medical professional, e.g., a practicing physician, dentist or podiatist. First, it is important that the face mask and its associated tubing do not impede access by the dentist or other medical professional to the person's mouth. Anesthesia masks typically used in general surgery cover both the nose and mouth, and include tubes that extend into the patient's throat. Such mask systems cannot be used when access to the patient's mouth is required. Second, the patient may exhale significant quantities of the anesthetic, which can then enter the atmosphere of the dental office where it can be inhaled by dentists and dental assistants. Therefore such systems also may include means for recovering the exhaled anesthetic gas so that the gas does not enter the general atmosphere of the office. However such gas recovery systems may be cumbersome. It is important that such gas recovery systems do not block or impede access to the person's mouth. Third, in some cases the medical professional may administer a blend of oxygen and an anesthetic gas, or may administer oxygen alone. It therefore would be desirable to provide a system that could administer only oxygen, or only gaseous anesthesia, or a mixture of oxygen and gaseous anesthesia. Fourth, it is desirable to monitor the gases being exhaled by the patient; in particular the medical professional may wish to monitor the level of exhaled carbon dioxide. It therefore would be desirable to provide a means to sample the gases exhaled by the individual receiving gaseous treatment during the course of the medical procedure. In addition, it would be desirable to provide a face mask and anesthesia administration system that is simple to manufacture, is easy for the medical professional, e.g., an anesthesiologist, physician, dentist, or podiatrist, to operate, and is comfortable for the person receiving anesthesia.

### SUMMARY OF THE INVENTION

These and other objects of the invention are met by the face mask and anesthesia administration system disclosed herein. A face mask is provided, the face mask being configured for placement over the nose but not the mouth of a person. The face mask has an inner surface and an outer surface, such that when the face mask is placed over the person's nose a nasal breathing plenum is defined between the person and the inner surface of the face mask in the region surrounding the person's nose. The face mask includes a gas inlet means, a gas outlet means, and one or more, preferably one, gas sample access means; preferably the gas inlet means, gas outlet means, and gas sample access means are each integrally formed with the mask in a one-piece construction. The gas inlet means is connectable to a source of gas to be administered to the person. The gas outlet means is connectable to a means for removing gas from the nasal breathing plenum. The gas sample access means is connectable to a system that can analyze a gas sample for carbon dioxide content, or other components of possible interest. The face mask is advantageously configured so that in use the connections to the gas source, the gas removal system and the gas sample analyzing system are each oriented away from the person's mouth where the medical professional will be working. The face mask may also be disposable, so that the mask may be a single-use product.

In operation, the gas mask is placed over the patient's nose, and connected to a source of gas to be administered. If gaseous anesthesia is to be included in the gas to be administered, the face mask also will be connected to a gas removal system. The gas is then administered to the patient, and the medical professional proceeds with the medical or dental procedure in the person's mouth. During the procedure the gas in the nasal breathing plenum can be accessed, if desired, to monitor the composition of the gas, such as for carbon dioxide or other components of interest.

The gas mask of the invention is versatile and permits monitoring of gas in the nasal breathing plenum and administration of various gases to the patient as needed. For example, the patient may be administered supplemental oxygen alone or a mixture of oxygen and nitrous oxide. In addition, the gas mask may be swapped out for an anesthesia mask (covering both the nose and mouth) should the patient require such treatment.

### BRIEF DESCRIPTION OF THE DRAWINGS

The concept of the present invention may be more readily understood by reference to the drawings herein, wherein like reference numerals indicate like parts, and wherein

FIG. 1 is a perspective view of a face mask for use in the present invention;

FIG. 2 is a front elevation view of a face mask for use in the present invention;

FIG. 3 is a rear elevation view of a face mask for use in the present invention;

FIG. 4 is a right side elevation view of a face mask for use in the present invention;

FIG. 5 is a left side elevation view of a face mask for use in the present invention;

FIG. 6 is a top plan view of a face mask for use in the present invention;

FIG. 7 is a bottom plan view of a face mask for use in the present invention;

FIG. 8 is a perspective view of a face mask for use in the present invention, showing the mask with connections to a gas source and a gas removal system, and further showing a means for securing the mask to an individual;

FIG. 9 is an exploded perspective view of the face mask for use in the present invention; and

FIG. 10 is a perspective view of a face mask for use in the present invention shown in use on a patient.

FIGS. 11A and 11B are perspective views of a face mask for use in the present invention;

FIGS. 12A-12C are front elevation views of a face mask for use in the present invention;

FIG. 13A is a rear elevation view of a face mask for use in the present invention;

FIG. 13B is a rear elevation view of a face mask for use in the present invention;

FIG. 14 is a right side elevation view of a face mask for use in the present invention;

FIG. 15 is a left side elevation view of a face mask for use in the present invention;

FIG. 16A is a top plan view of a face mask for use in the present invention;

FIG. 16B is a top plan view of a face mask for use in the present invention;

FIG. 17 is a bottom plan view of a face mask for use in the present invention;

FIG. 18 is a perspective view of a face mask for use in the present invention, showing the mask with connections to a gas source and a gas removal system, and further showing a means for securing the mask to an individual;

FIG. 19 is an exploded perspective view of the face mask for use in the present invention; and

FIG. 20 is a perspective view of a face mask for use in the present invention shown in use on a patient.

### DETAILED DESCRIPTION OF THE INVENTION

As illustrated in the figures, a face mask 10 for use in the present invention comprises a cup-shaped shell 12 having a curved wall 14 terminating at a periphery 16. Curved wall 14 of shell 12 is configured to fit over a person's nose while providing access to a person's mouth. Shell 12 is provided about the periphery 16 of curved wall 14 with a flexible peripheral seal 20. Seal 20 has a forward edge 22 that engages the periphery 16 of curved wall 14 in substantially air-tight engagement, and a rearward edge 24 that engages the face of the person to whom the gas is administered. Seal 20 preferably is made of a material that has sufficient flexibility and resiliency to provide a comfortable seal against the person's face, while having sufficient firmness to support shell 12 in operable relation to the person being treated. The inner surface of seal 20, the inner surface of curved wall 14, and the surface of the person's face when the mask is applied together define a region in space referred to herein as the nasal breathing plenum.

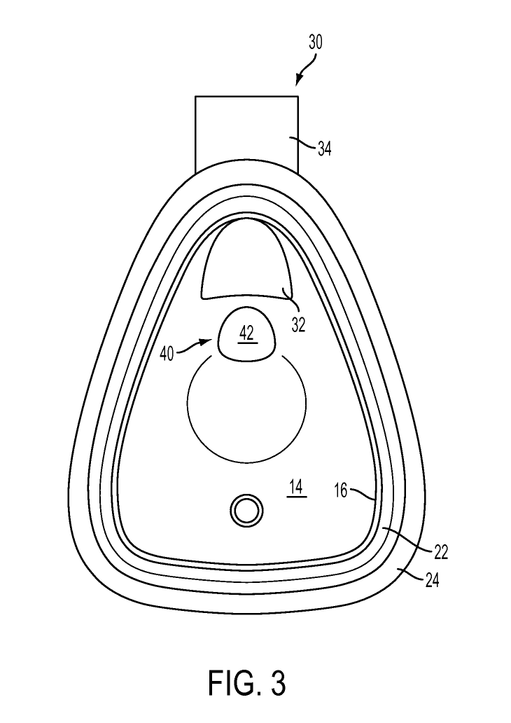

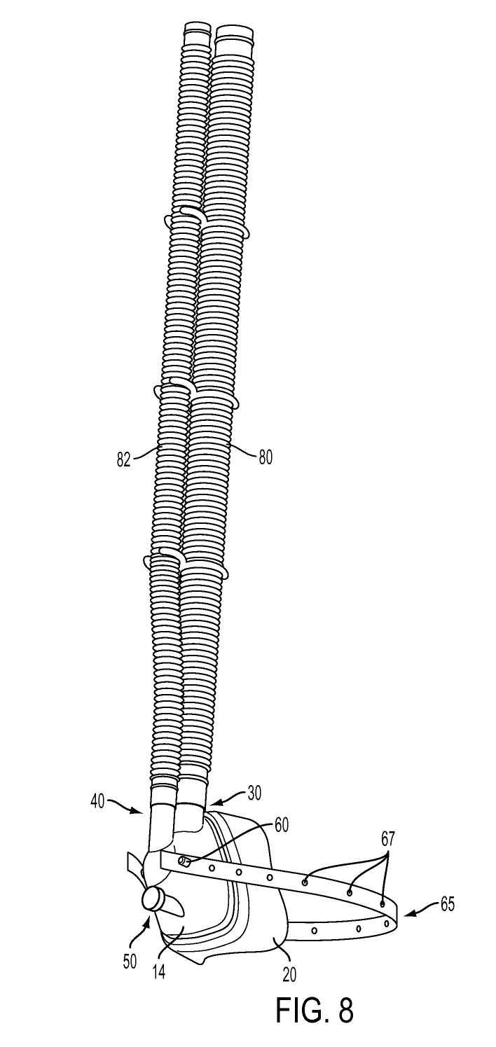

Curved wall 14 of shell 12 is provided with gas inlet means 30 comprising gas inlet port 32 (FIG. 3) from which extends gas inlet channel 34. Gas inlet channel 34 is advantageously sized and dimensioned to allow for connection to standard connectors typically used with oxygen and anesthesia tubing, such as gas inlet tubing 80 (FIG. 8). For example, in one embodiment gas inlet channel 34 may have an outer diameter of about 15 mm. Gas inlet means 30 can be used to introduce any desired gas from a gas source. The gas can be oxygen, an anesthetic such as nitrous oxide, or a mixture of the two, as will be known to those skilled in the art Gas inlet tubing 80 is connected at one end to gas inlet channel 34, and at its other end to a gas source as in well known in the art (not shown). In a preferred embodiment, gas inlet channel 34 has no bends or elbows that would block or impede the flow of gas from the tubing 80 through inlet port 32 in curved wall 14 of shell 12 to reach the person being treated.

Where gas inlet channel 34 has an outer diameter of, for example, about 15 mm, it may be connected to gas inlet tubing having a 15 mm ID opening at one end (the mask end) and a dimension at its distal end appropriate for connection to standard connectors to a gas source. For example, in one embodiment, the distal end of the gas inlet tube has an inner diameter of about 22 mm and may be connected to a gas source having a appropriately dimensioned supply connector.

In certain embodiments, there will be multiple gas sources available to the professional, e.g., oxygen and nitrous oxide, preferably rapidly interchangeable from one gas supply to another, or to a blend of gases, via, for example, appropriate shut-off valves.

In certain situations, it may be necessary to administer full anesthesia to the patient, in which case the gas mask may be swapped out for an anesthesia mask that covers both the nose and the mouth and that has a gas inlet channel of the same diameter as the gas inlet channel 34 of the inventive mask.

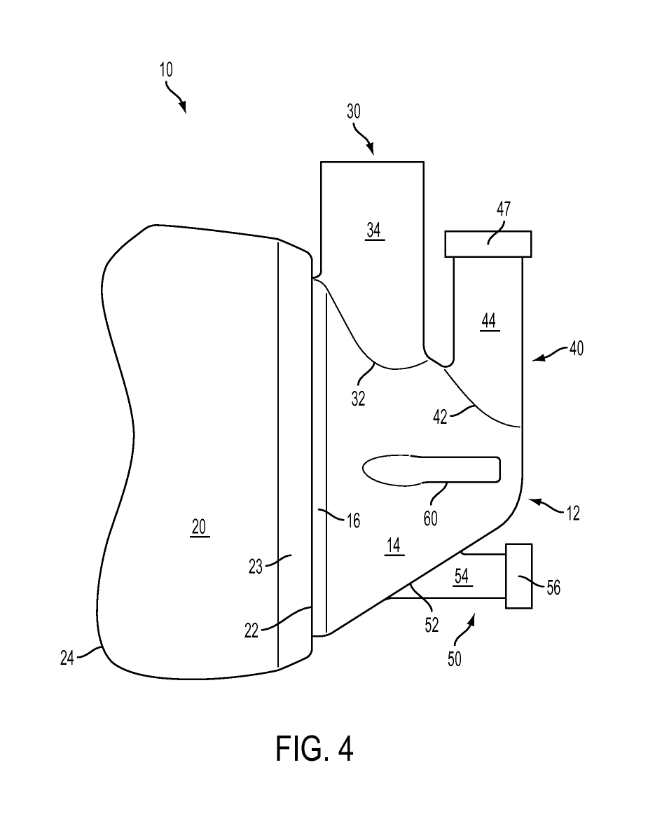

Curved wall 14 of shell 12 is further provided with gas outlet means 40 comprising gas outlet port 42 from which extends gas outlet channel 44. Gas outlet channel 44 is advantageously sized and dimensioned to allow for connection to standard tubing that will convey gases to a gas recovery system if desired. Gas outlet means 40 may be sized somewhat smaller than gas inlet means 30, e.g., about 10 mm outside diameter (OD), to facilitate a positive pressure within the nasal breathing plenum define by mask 10. If the gas being administered is only oxygen, then there will be no need to recover the exhaled gases, in which case the gas outlet channel can be provided with a cap 47 (FIG. 4), plug, or similar stopper. Alternatively, if the gas administered to the person includes gaseous anesthesia, then gas outlet channel can be connected to a gas outlet tubing 82 that can convey gas from the nasal breathing plenum to a gas recovery means such as are known in the art. In a preferred embodiment, gas outlet channel 44 has no bends or elbows that would block or impede the flow of gas from the nasal breathing plenum to the outlet tubing 82.

It is an advantage of the present invention that shell 12 does not include any vents, or perforations, that would allow the escape of anesthetic gases into the general atmosphere of the room in which the procedure is being performed, so that such gases will not affect other persons in the room. The non-vented or perforated shell also allows for the administration of positive pressure oxygen should the patient's oxygen saturation decrease.

It is another advantage of the present invention that gas inlet channel 34 and gas outlet channel 44 are oriented in such a manner that gas inlet tubing 80 and gas outlet tubing 82 are oriented away the patient's mouth and away from the working area of the medical professional performing the procedure on the patient. In the illustrated embodiment, the inlet channel 34 and the outlet channel 44 are oriented substantially vertically and on the top facing portion of shell 12, so that inlet tubing 80 and outlet tubing 82 are directed substantially vertically upward, away from the patient's mouth and away from the region where the medical professional is working.

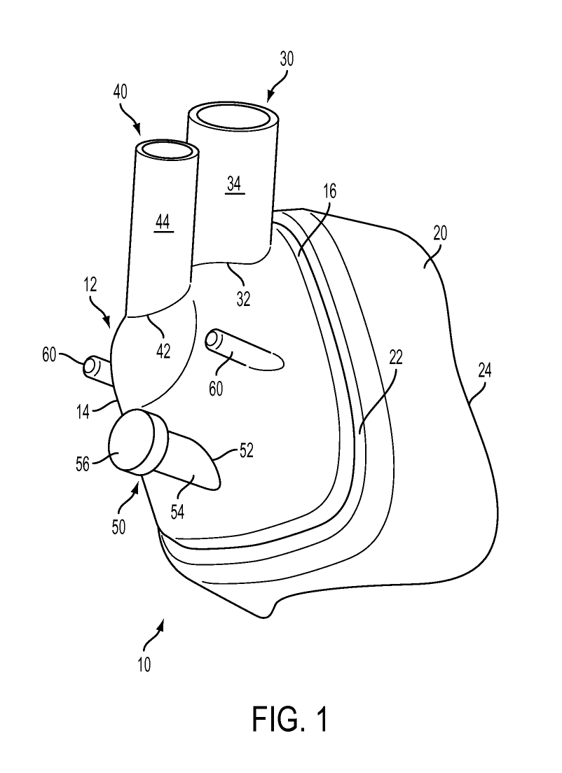

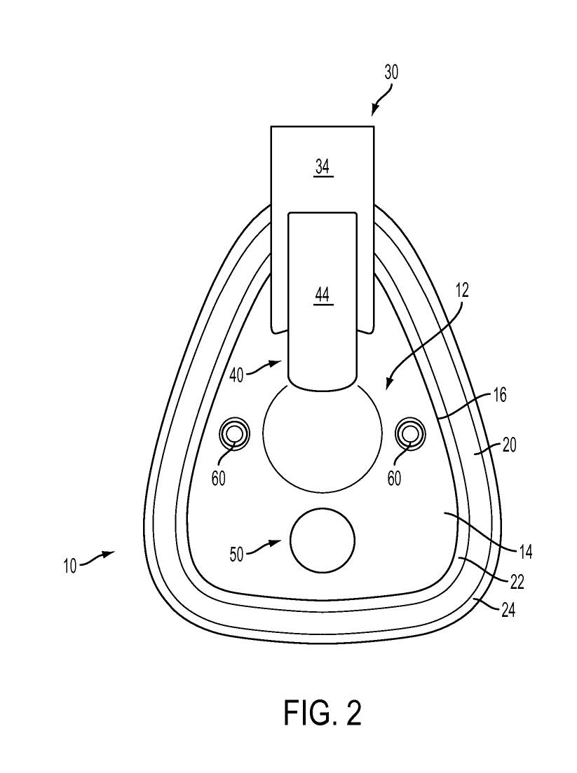

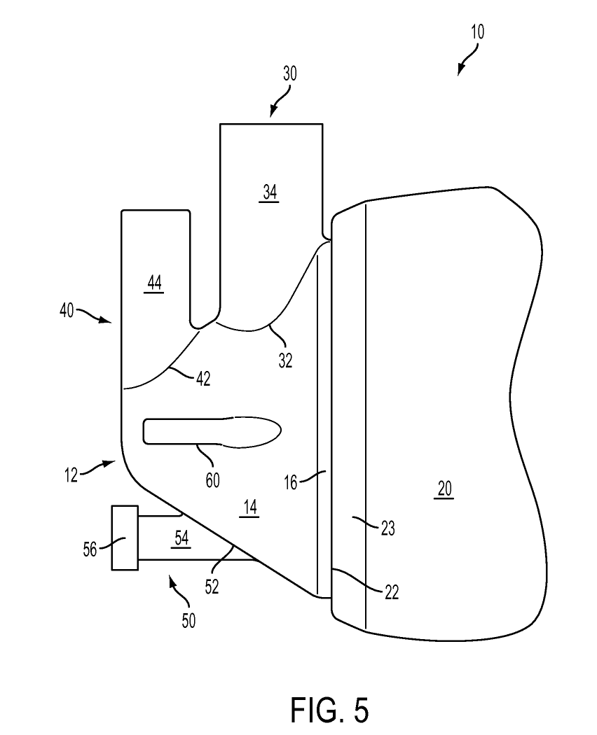

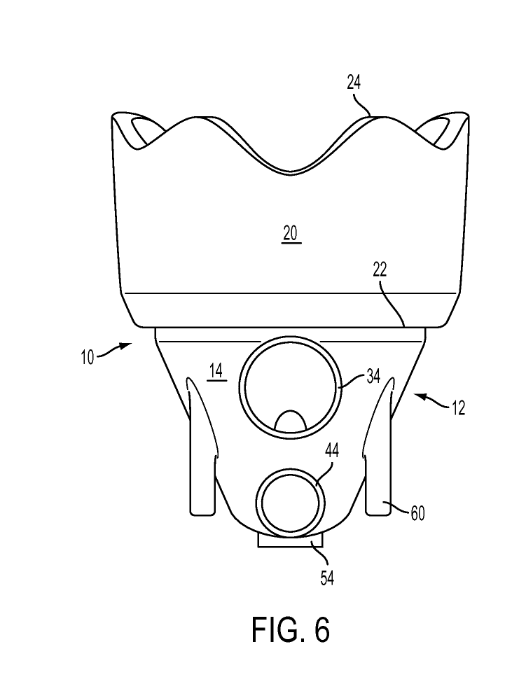

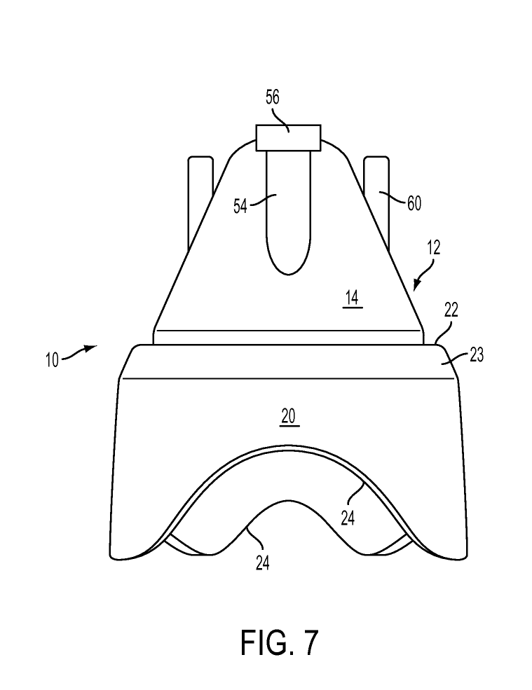

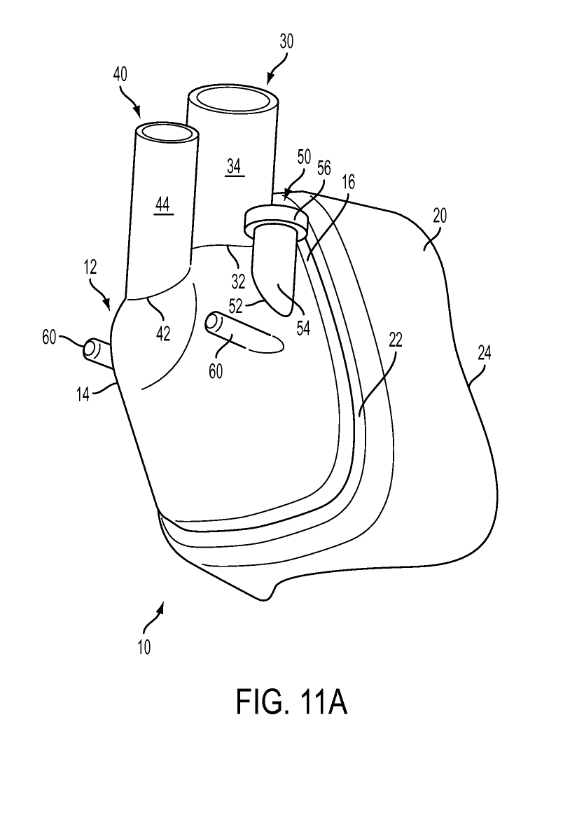

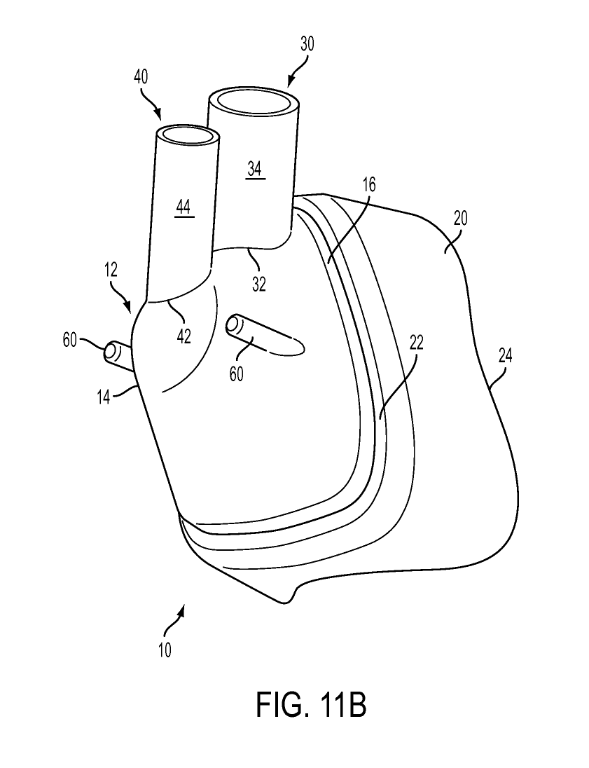

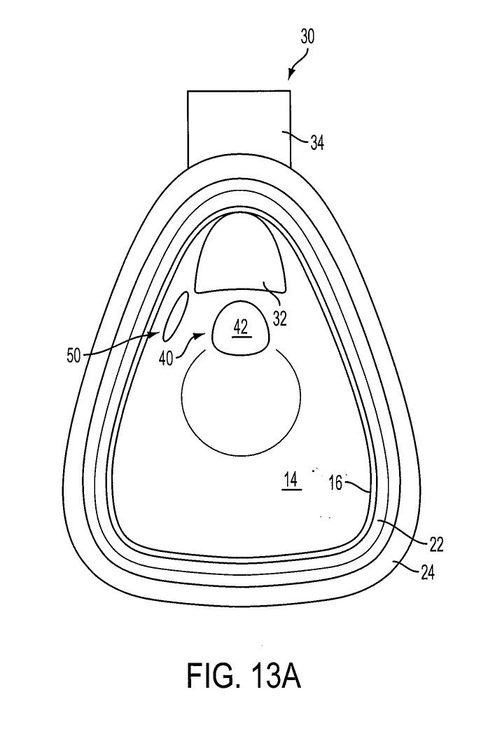

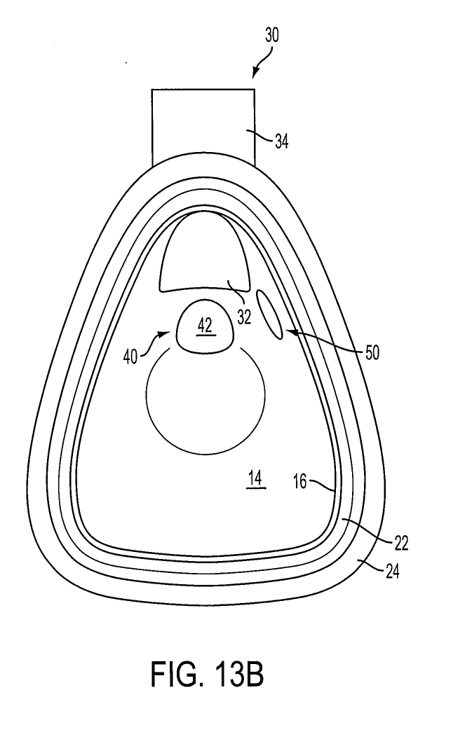

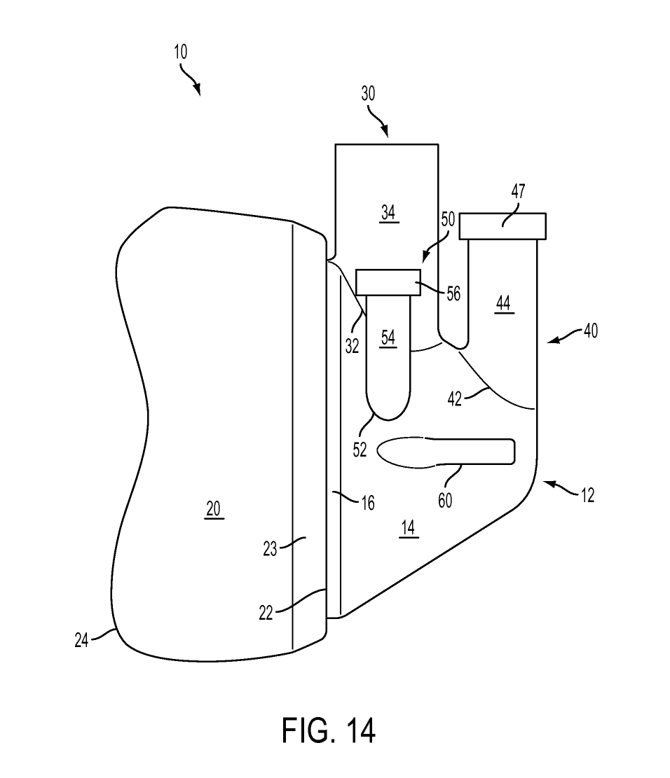

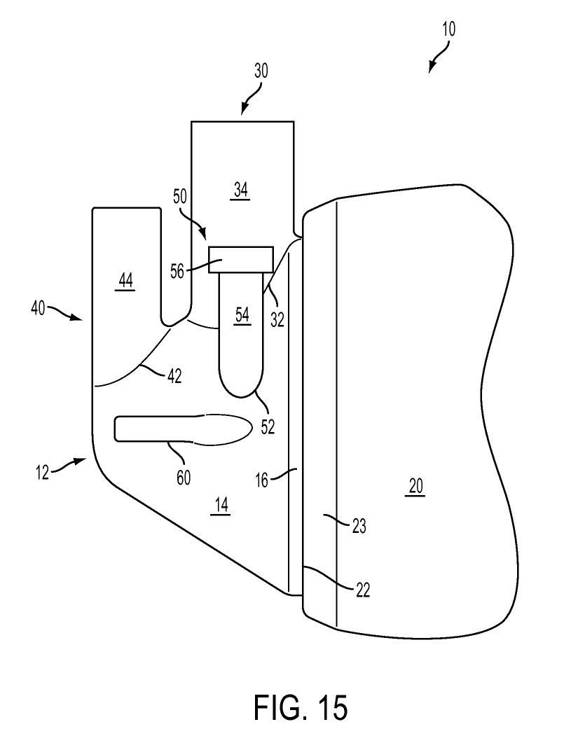

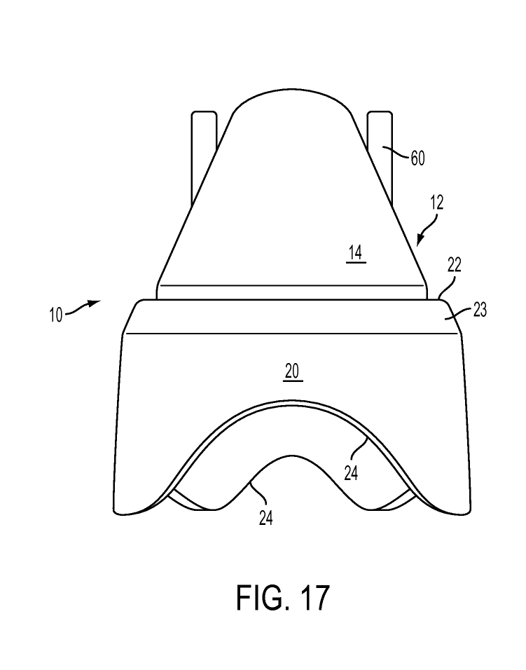

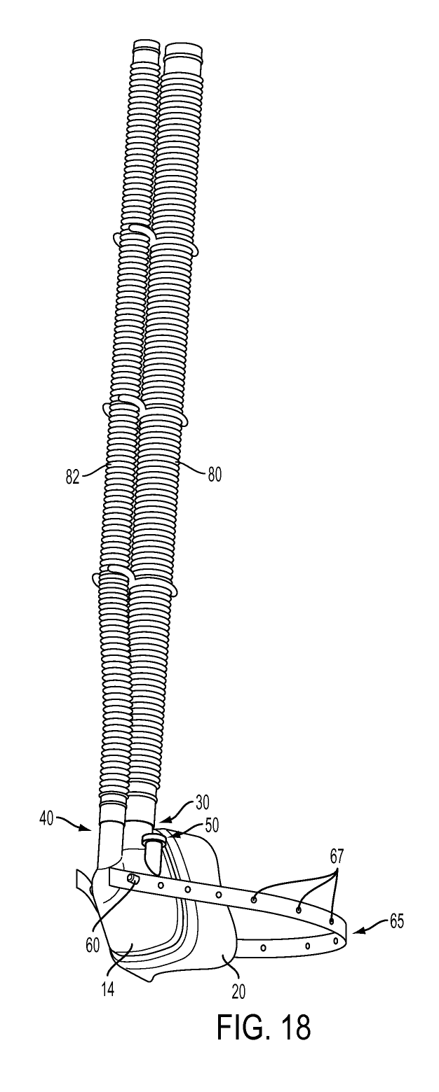

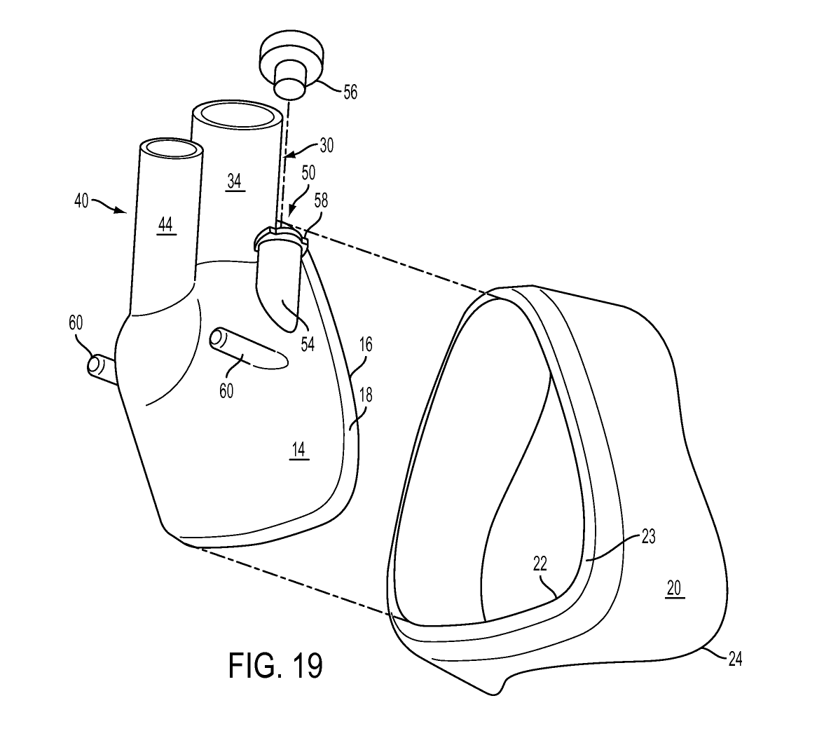

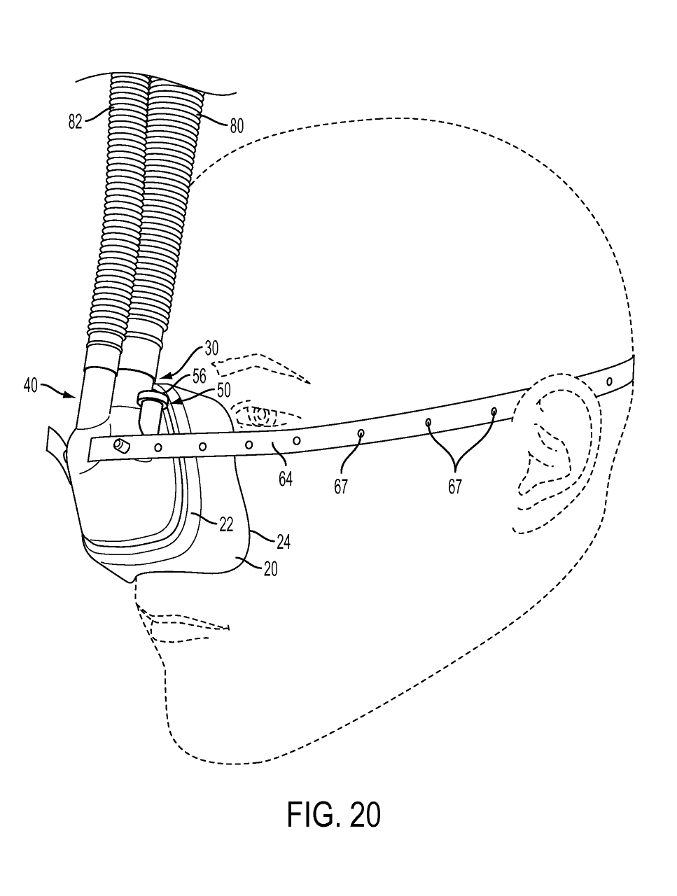

In a preferred embodiment, the mask 10 is further provided with a sampling access means 50 to allow sampling of the gas in the nasal breathing plenum. In the embodiment illustrated in FIGS. 1-10, curved wall 14 of shell 12 is provided with a sampling port 52 from which sample access channel 54 extends substantially horizontally, i.e., forward from the patient. In an alternative embodiment, as shown in FIGS. 11-20, curved wall 14 of shell 12 is provided with a sampling port 52 from which sample access channel 54 extends substantially vertically, i.e., in a direction that is substantially parallel to outlet channel 44 and inlet channel 34. Sampling port 52 and sampling access channel 54 can be significantly smaller in diameter than the gas inlet and outlet ports and the gas inlet and outlet channels, respectively. In a procedure in which sampling will not be performed, sampling access channel 54 can be provided with a cap 56 (FIG. 4) or other suitable closure means to maintain the pressure within the nasal breathing plenum. In a procedure in which sampling of the gas in the plenum will be performed, cap 56 is removed and a sampling tubing (not shown) can be affixed to the open end of sampling access channel 54. As is known in the art, such tubing can be provided with a valve which can be opened to allow sampling of the gas in the nasal breathing plenum when desired, and closed when sampling is not being done. Sampling channel 54 is positioned on curved wall 14 of shell 12 to facilitate easy access by the medical professional performing the procedure. At the same time, the short length of sample access channel 54 relative to gas inlet channel 34 and gas outlet channel 44 ensures that the sampling access channel 54 will not be in the medical professional's way as work is being performed in the person's mouth.

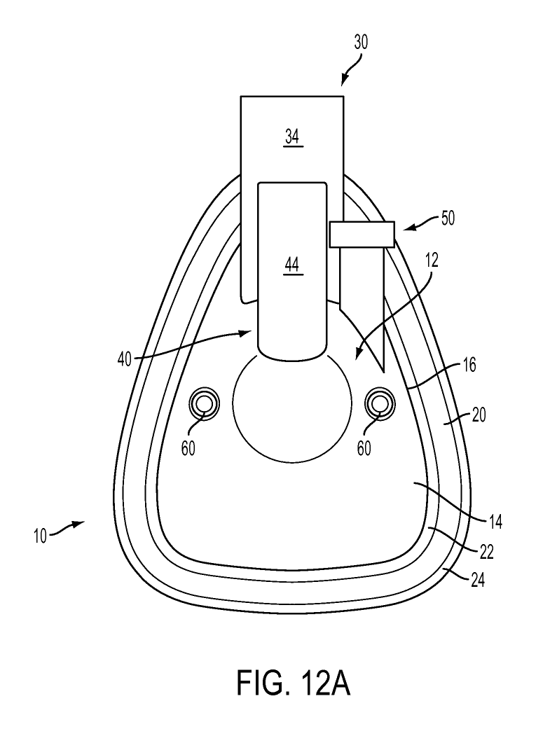

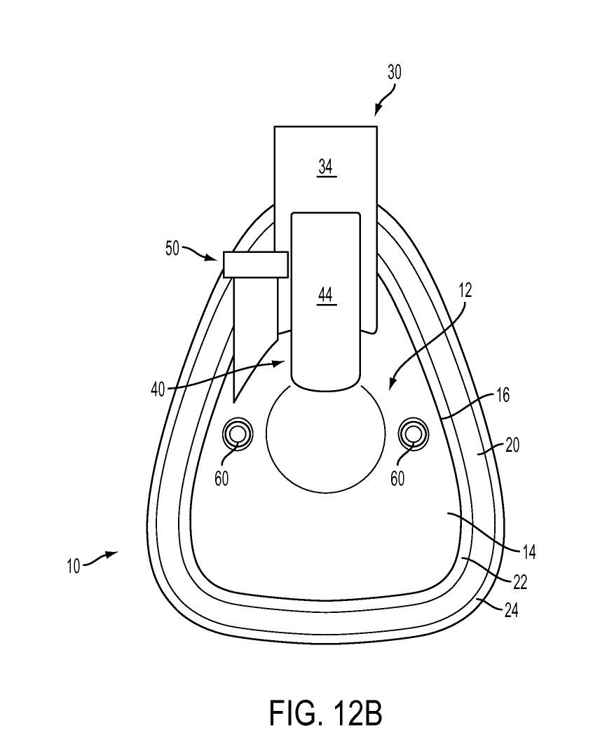

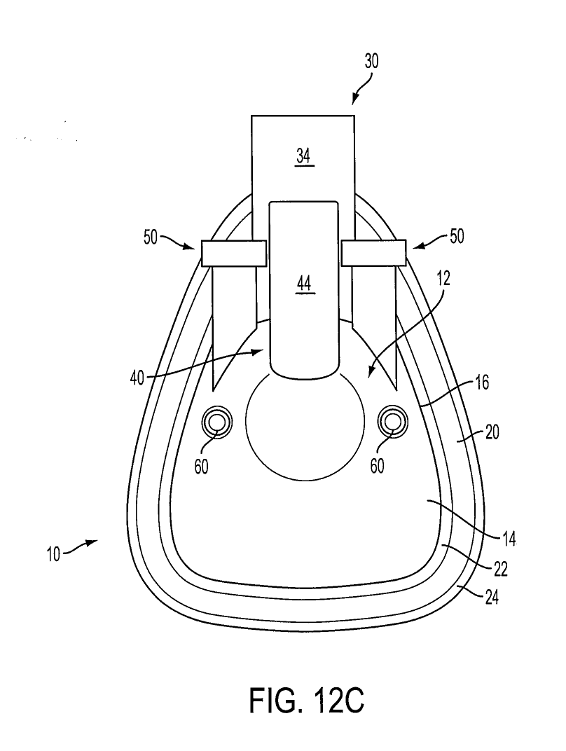

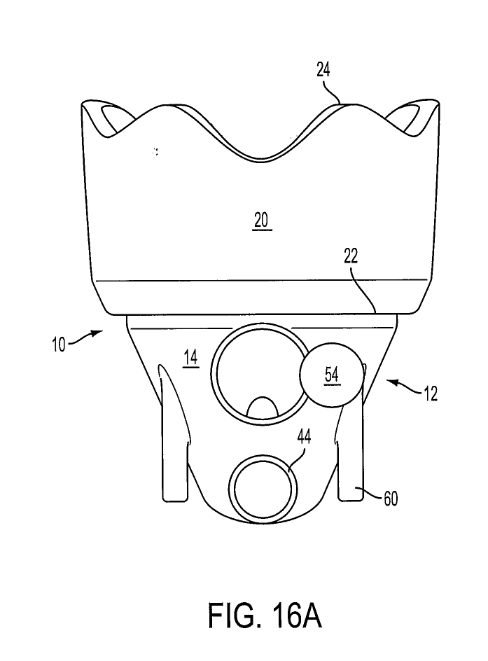

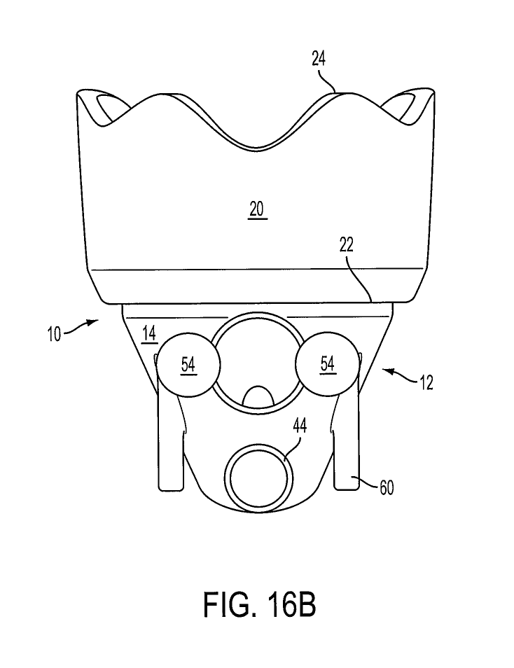

The embodiments shown in FIGS. 12C and 16B include more than one gas sample access means 50, where the channel 54 of access means 50 are depicted in FIG. 16B as capped with cap 56.

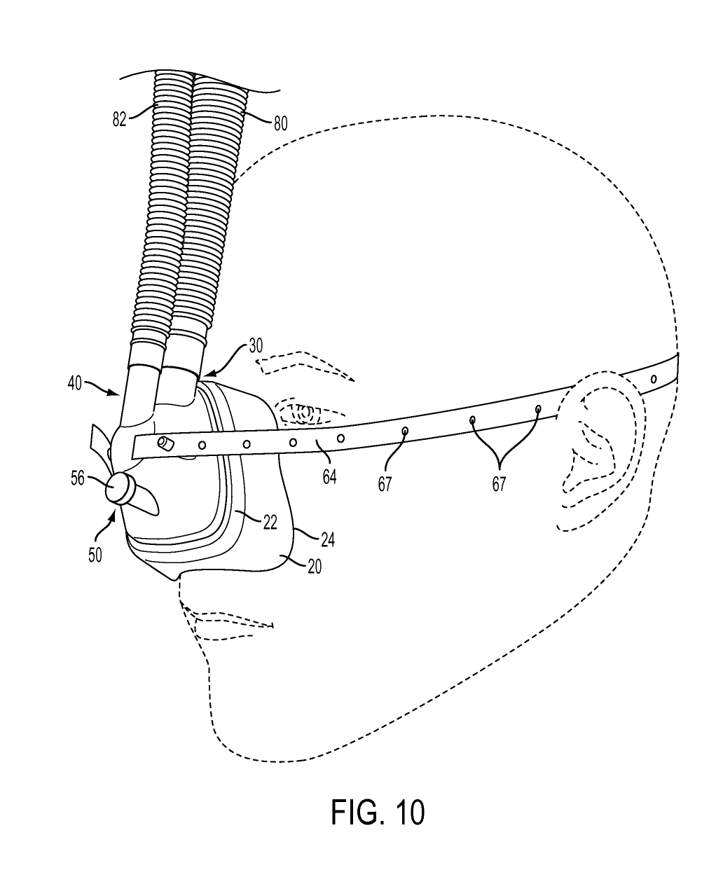

Mask 10 can be secured to the individual by means of a head strap such as is known in the art. To facilitate such securement, shell 14 is provided on the outer surface of curved wall 14 with one or more forwardly projecting lugs 60 to which a head strap may be secured. Advantageously two lugs 60 can be used, one on each side of curved wall 14. As shown in FIG. 10, strap 65 is provided with perforations 67. Strap 65 preferably is made of an elastic material. Selected perforations 67 of strap 65 are placed over lugs 60, and the strap 65 is placed against the back of the wearer's head, to provide a secure yet comfortable fit of mask 10 to the user's face.

Shell 12 of mask 10 is preferably a plastic material that can be formed into a desired shape by standard molding procedures such as injecting molding. The lack of any bends or elbows in any of the gas inlet channel, gas outlet channel, and sample access channel facilitates ease of injection molding and removal of the finished part from the mold. In a preferred embodiment shell 12 is transparent, more preferably colorless, so that the attending medical professional can monitor a patient's breathing by observing the appearance and disappearance of condensed breath vapors on the inner surface of shell 12. In addition, the design of the mask is free of valves or other moving parts that would complicate both manufacture and use of the mask

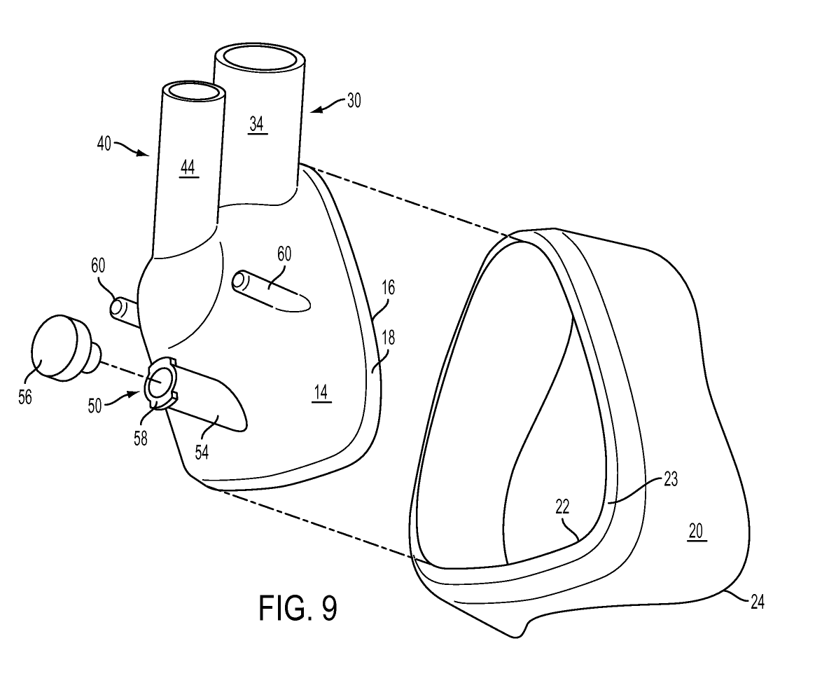

FIG. 9 is an exploded view of mask 10 to facilitate understanding of the assembly of shell 12, peripheral seal 20 and cap 56. Shell 12 includes curved wall 14 terminating at a periphery 16, the periphery 16 including integrally molded collar 18 that circumscribes periphery 16. Peripheral seal 20 has a forward edge 22 including a circumferential lip 23 adapted to engage molded collar 18 of shell 12 to provide a substantially air-tight seal therebetween. Peripheral seal 20 can be provided in larger and smaller sizes to better adapt the face mask 10 to the size of the person to be treated. Cap 56 fits across the open end of sample access channel 54, to which it may be removably secured by conventional securement means such as lateral tabs 58 that facilitate ease of removal and replacement of cap 56.

FIG. 10 illustrates a method of using the face mask of the present invention, in which the face mask 10 of the present invention is secured to a patient, shown in phantom lines. Face mask 10 covers the patient's nose to define a nasal breathing plenum between the inner surface of the mask and the patient. Face mask 10 is secured to the patient by strap 65, which extends behind the patient's head and is removably secured to face mask 10 by placing perforations 67 over forwardly projecting lugs 60. Gas to be administered to the patient passes through gas inlet tubing 80 through gas inlet channel 34 and gas inlet port 32 of gas inlet means 30 to reach the nasal breathing plenum between the inner surface of face mask 10 and the patient's face. If the gas administered is oxygen or another gas that can be released to the environment, then gas outlet channel can be provided with cap 47. If the gas provided to the patient includes a gaseous anesthetic or other gas which should not be released to the environment, then gas outlet channel 44 will be connected to gas outlet tubing 82, such that gas in the nasal breathing plenum which should not be released to the environment will pass through gas outlet port 42 of gas outlet means 40, through gas outlet 44 channel and into gas outlet tubing 82, from which it can be conveyed to a gas scavenger system or other gas containment system such as is known in the art. The rate of flow of gas into the face mask 10, the rate of flow out of the face mask 10, and the relative sizes of the inlets and outlets will be set and configured so as to remain a positive pressure of gas within the nasal breathing plenum. It will be observed that the face mask 10 of the present invention does not require valves at the gas inlet and outlet ports, and does not require cannulae to be inserted into the nares of the patient, thereby facilitating patient comfort and compliance with the system. In addition, the face mask 10 affords the medical professional ready access to the patient's mouth for dental or other procedures, and orients he inlet and outlet tubing 80, 82 vertically upward to be out of the way during the course of the procedure

If it is desired to sample the gas within the nasal breathing plenum, such as to monitor the level of carbon dioxide or other gases, then cap 56 can be removed from sampling access channel 54, and standard tubing supplied with a luer lock mechanism can be affixed to sampling access channel 54 to allow samples of the gas in the plenum to be transported to a gas analyzer (not shown) as is known in the art.

### Parts List

10—face mask
12—cup-shaped shell
14—curved wall
16—periphery of curved wall
18—molded collar circumscribing periphery 16
20—peripheral seal
22—forward edge of peripheral seal
23—circumferential lip on forward edge 22 of seal 20
24—rearward edge of peripheral seal
30—gas inlet means
32—gas inlet port
34—gas inlet channel
40—gas outlet means
42—gas outlet port
44—gas outlet channel
47—cap for gas outlet channel
50—sampling access means
52—sampling port
54—sampling access channel
56—cap for sampling access channel
58—lateral tabs for securement of cap 56
60—forwardly projecting lugs
65—strap
67—perforations
80—gas inlet tubing
82—gas outlet tubing

What is claimed is:

1. An anesthesia administration system for administering a gaseous anesthesia to a person in need thereof, the administration system comprising
a) a face mask having an inner surface and an outer surface, said face mask being configured for placement over the nose but not the mouth of the person receiving anesthesia to define a nasal breathing plenum between the person's nose and the inner surface of said face mask, the face mask having an integrally formed gas inlet means, and an integrally formed gas outlet means, and an integrally formed gas sample access means;
b) means for delivering a gaseous anesthesia to said nasal breathing plenum through said integrally formed gas inlet means;
c) means for removing gas in said nasal breathing plenum through said integrally formed gas outlet means; and
d) means for accessing said integrally formed gas sample access means;
whereby said anesthesia administration system is suitable for use in administering gaseous anesthesia to an individual undergoing a medical or dental procedure, while simultaneously allowing sampling of the gas within said nasal breathing plenum.

2. The anesthesia administration system of claim 1 wherein said integrally formed gas inlet means is substantially straight.

3. The anesthesia administration system of claim 2 wherein said integrally formed gas inlet means is substantially vertical relative to the orientation of said face mask.

4. The anesthesia administration system of claim 1 wherein said integrally formed gas outlet means is substantially straight.

5. The anesthesia administration system of claim 4 wherein said integrally formed gas outlet means is substantially vertical relative to the orientation of said face mask.

6. The anesthesia administration system of claim 1 wherein said integrally formed gas sample access means is directed substantially forward of the patient receiving said anesthesia.

7. The anesthesia administration system of claim 1 being operable in the absence of one or more cannulae for delivering said gaseous anesthesia into the nostrils of the patient.

8. The anesthesia administration system of claim 1 wherein the face mask does not include valves.

9. The anesthesia administration system of claim 1, wherein the gas sample access means is substantially vertical relative to the orientation of said face mask

10. The anesthesia administration system of claim 1, wherein the gas outlet means is sized smaller than gas inlet means.

11. A device for use in administering gaseous anesthesia to a patient in need thereof, the device comprising pumping gas anesthesia gas through
a face mask having an inner surface and an outer surface, said face mask being suitable for placement over the nose but not the mouth of the person receiving anesthesia to define a nasal breathing plenum between the person's nose and the inner surface of said face mask, the face mask having an integrally formed gas inlet means, an integrally formed gas outlet means, and an integrally formed gas sample access means;
wherein said face mask can be operatively coupled to a gas delivery and removal system, whereby gaseous anesthesia can be delivered to said patient through said gas inlet means and removed through said gas outlet means, while maintaining a positive gas pressure at the patient's nose.

12. A method for administering gaseous anesthesia to a patient in need thereof and under positive gas pressure at the patient's nose, the method comprising supplying gaseous anesthesia under pressure via gas inlet tubing to a face mask,
wherein
the face mask has an inner surface and an outer surface, said face mask being suitable for placement over the nose but not the mouth of the patient receiving anesthesia to define a nasal breathing plenum between the patient's nose and the inner surface of said face mask, the face mask having an integrally formed gas inlet means, an integrally formed gas outlet means, and an integrally formed gas sample access means;
where the gas outlet means is sized smaller than gas inlet means; and
said face mask being capable of operative coupling to a gas delivery and removal system.
